(19) United States
(12) Patent Application Publication
(10) Pub. No.: US 2025/0124188 A1
(43) Pub. Date: Apr. 17, 2025

(54) METHOD OF DESIGNING A CUSTOMIZED FOOT ORTHOSIS

(71) Applicant: CRYOS TECHNOLOGIES INC., Joliette (CA)

(72) Inventors: Gregory Nicholas WATTS, Joliette (CA); Philippe LEGARE, Joliette (CA); Frederic GREMILLET, Joliette (CA); Caroline GEORGE, Joliette (CA); Etienne OUELLETTE, Joliette (CA)

(21) Appl. No.: 18/688,131

(22) PCT Filed: Sep. 2, 2022
(86) PCT No.: PCT/CA2022/051325

§ 371 Date: Feb. 29, 2024

Related U.S. Application Data
(60) Provisional application No. 63/260,887, filed on Sep. 3, 2021.

(51) Int. Cl. G06F 30/20; A61F 5/14

(52) U.S. Cl. CPC G06F 30/20; A61F 5/14

(57) ABSTRACT

A method of designing a foot orthosis includes generating a customized digital model by modifying an initial digital model of the orthosis to include a posting structure having a surface profile obtained by applying local displacements to surface points of the initial model. Generating the customized model can include determining a peripheral and a salient portion of the surface profile from first and second subsets of the surface points, respectively; determining, for each point, a value of a first displacement parameter representative of a position of the point relative to the peripheral and salient portions, the peripheral and salient portions being respectively associated with minimum and maximum values of the first parameter, and a value of a second displacement parameter representative of a distance of the surface point from a reference surface; and computing, for each point, the local displacement based on values of the first and second parameters.

### RELATED PATENT APPLICATION

The present application claims priority to U.S. Provisional Patent Application No. 63/260,887 filed on Sep. 3, 2021, the disclosure of which is incorporated by reference in its entirety.

### TECHNICAL FIELD

The technical field generally relates to foot orthotics and, more particularly, to a method of designing a customized foot orthosis including a posting structure, which can be fabricated using additive manufacturing.

### BACKGROUND

Orthotic inserts, also referred to as foot orthoses or orthotics, are a type of medical device used to support the foot of a patient. They are used to correct for biomechanical faults, for example, if a patient runs or walks in a way that may cause pain or harm posture. The correction can be provided by improving foot support, for example in a medial (inside) or lateral (outside) portion of the foot. Orthotic inserts can be augmented by other components, such as a postings, to improve foot support. Conventionally, a posting is manually added to the heel portion of the orthotic insert to provide medial or lateral support and achieve a corresponding correction to the patient. Postings are generally made separate from the orthotic insert itself, and then attached thereto, resulting in additional manufacturing time and complexity, as well as added inventory management. Existing manufacturing practices also result in postings having less than ideal shapes.

### SUMMARY

The present description generally relates to computer-aided techniques for designing and manufacturing customized foot orthoses.

In accordance with an aspect, there is provided a method of designing an orthosis for a foot of a patient, the method including:
providing an initial digital model of the orthosis; and
generating a customized digital model of the orthosis by modifying the initial digital model to include a posting structure, the posting structure having a surface profile obtained by applying a set of local surface displacements to a corresponding set of surface points of the initial digital model, said generating including:
   determining a peripheral portion of the surface profile of the posting structure, the peripheral portion being formed from a first subset of the set of surface points of the initial digital model;
   determining a salient portion of the surface profile of the posting structure, the salient portion being formed from a second subset of the set of surface points of the initial digital model;
   determining, for each surface point, a local value of a first displacement parameter, the first displacement parameter being representative of a position of the surface point with respect to the peripheral portion and the salient portion of the surface profile of the posting structure, wherein the first subset of surface points is associated with a minimum value of the first displacement parameter and the second subset of surface points is associated with a maximum value of the first displacement parameter;
   determining, for each surface point, a local value of a second displacement parameter, the second displacement parameter being representative of a distance of the surface point from a reference surface associated with the initial digital model; and computing, for each surface point, the local surface displacement based on the local value of the first displacement parameter and the local value of the second displacement parameter, thereby generating the customized digital model.

In one embodiment, determining, for each surface point, the local value of the first displacement parameter includes determining an initial value of the first displacement parameter; and applying a smoothing function to the initial value to obtain the local value of the first displacement parameter.

In one embodiment, the smoothing function is a sigmoid curve.

In one embodiment, determining, for each surface point, the initial value of the first displacement parameter includes determining a distance ratio of the shortest distance of the surface point from the peripheral portion to a sum of the shortest distance of the surface point from the peripheral portion and the shortest distance of the surface point from the salient portion; and using the distance ratio as the initial value of the first displacement parameter.

In one embodiment, the reference surface associated with the initial digital model is a ground plane in contact with a ground-contacting region of the initial digital model.

In one embodiment, determining, for each surface point, the local value of the second displacement parameter includes determining a distance of the surface point from the ground plane; and setting the local value of the second displacement parameter equal to a fraction of the distance of the surface point from the ground plane.

In one embodiment, the method includes providing a virtual library containing a plurality of pre-set posting height percentages and selecting, from the virtual library, a selected one of the plurality of pre-set posting height percentages as the fraction of the distance of the surface point from the ground plane.

In one embodiment, the selected pre-set posting height percentage is the same for all the surface points of the set of surface points of the initial digital model.

In one embodiment, the peripheral portion of the surface profile of the posting structure coincides, along a portion thereof, with an outer contour of the initial digital model of the orthosis.

In one embodiment, the peripheral portion of the surface profile of the posting structure is entirely inwardly offset from an outer contour of the initial digital model of the orthosis.

In one embodiment, the posting structure is formed on a heel region of the initial digital model.

In one embodiment, determining the peripheral portion of the surface profile of the posting structure includes determining a rear section of the peripheral portion; and determining a front section of the peripheral portion based on the rear section.

In one embodiment, providing the initial digital model of the orthosis includes forming a heel notch in the heel region to split a rear contour of the heel region into a medial segment and a lateral segment; and determining the rear section of the peripheral portion includes forcing the rear section to pass along part of the heel notch and along part of either the lateral or the medial segment, or both the lateral segment and the medial segment.

In one embodiment, forming the heel notch includes determining, a rearmost point, a medial point, and a lateral point in the heel region of the initial digital model; connecting the rearmost point to the medial point with a curve to generate the rear contour of the heel region; and generating the heel notch through the curve to split the rear contour of the heel region into the lateral segment and the medial segment.

In one embodiment, the curve is a parabola.

In one embodiment, the front section of the peripheral portion is determined using a non-uniform rational basis spline (NURBS) curve defined by a set of control points, the set of control points including a first control point and a second control point corresponding to a first endpoint and a second endpoint of the rear section, respectively.

In one embodiment, the peripheral portion of the posting structure is partially coincident with a midline formed through the initial digital model of the orthosis.

In one embodiment, the peripheral portion of the posting structure extends laterally and medially of a midline formed through the initial digital model of the orthosis.

In one embodiment, the salient portion of the posting structure extends laterally and medially of the midline.

In one embodiment, the peripheral portion of the posting structure includes a straight line, an arc segment, a NURBS, or a combination thereof, passing through the midline.

In one embodiment, the salient portion of the surface profile of the posting structure is determined based on the peripheral portion.

In one embodiment, determining the salient portion of the surface profile of the posting structure includes determining a frontmost point and a rearmost point of the peripheral portion of the surface profile of the posting structure; creating a line extending between the frontmost point and the rearmost point of the peripheral portion; and defining the salient portion to correspond at least in part to an intermediate portion of the line.

In one embodiment, the method includes an additive manufacturing operation of fabricating the orthosis based on the customized digital model.

In one embodiment, the method includes providing foot image data of the foot of the patient, and wherein providing the initial digital model of the orthosis includes determining the initial digital model based on the foot image data.

In one embodiment, providing the foot image data includes scanning the foot to capture the foot image data as a topographical surface map of the foot.

In accordance with another aspect, there is provided a non-transitory computer readable storage medium having stored thereon computer readable instructions that, when executed by a processor, cause the processor to perform a method of designing an orthosis for a foot of a patient such as disclosed herein. For example, the method of designing the orthosis can include steps of providing an initial digital model of the orthosis; and generating a customized digital model of the orthosis by modifying the initial digital model to include a posting structure, the posting structure having a surface profile obtained by applying a set of local surface displacements to a corresponding set of surface points of the initial digital model, said generating including: determining a peripheral portion of the surface profile of the posting structure, the peripheral portion being formed from a first subset of the set of surface points of the initial digital model; determining a salient portion of the surface profile of the posting structure, the salient portion being formed from a second subset of the set of surface points of the initial digital model; determining, for each surface point, a local value of a first displacement parameter, the first displacement parameter being representative of a position of the surface point with respect to the peripheral portion and the salient portion of the surface profile of the posting structure, wherein the first subset of surface points is associated with a minimum value of the first displacement parameter and the second subset of surface points is associated with a maximum value of the first displacement parameter; determining, for each surface point, a local value of a second displacement parameter, the second displacement parameter being representative of a distance of the surface point from a reference surface associated with the initial digital model; and computing, for each surface point, the local surface displacement based on the local value of the first displacement parameter and the local value of the second displacement parameter, thereby generating the customized digital model.

In accordance with another aspect, there is provided a computer device including a processor and a non-transitory computer readable storage medium such as disclosed herein, the non-transitory computer readable storage medium being operatively coupled to the processor.

In accordance with another aspect, there is provided an additive manufacturing system for designing an orthosis for a foot of a patient, the additive manufacturing system including an additive manufacturing device; and a computer device such as disclosed herein, wherein the computer device is operatively coupled to the additive manufacturing device and configured to control the additive manufacturing device to manufacture the orthosis based on the customized digital model.

In one embodiment, the additive manufacturing system includes an imaging device operatively coupled to the computer device, the imaging device being configured to acquire foot image data of the foot of the patient, wherein the computer device is configured to receive the foot image data and provide therefrom the initial digital model of the orthosis.

It is appreciated that other method and process steps may be performed prior, during or after the steps described herein. The order of one or more steps may also differ, and some of the steps may be omitted, repeated, and/or combined, as the case may be. It is also to be noted that some steps may be performed using various analysis and processing techniques, which may be implemented in hardware, software, firmware, or any combination thereof.

Other objects, features, and advantages of the present description will become more apparent upon reading of the following non-restrictive description of specific embodiments thereof, given by way of example only with reference to the appended drawings. Although specific features described in the above summary and in the detailed description below may be described with respect to specific embodiments or aspects, it should be noted that these specific features may be combined with one another unless stated otherwise.

### BRIEF DESCRIPTION OF THE DRAWINGS

FIGS. 1A to 1C illustrate two faulty foot postures requiring an orthotic device (FIGS. 1A and 1C), in comparison to a third, normal posture (FIG. 1B);

FIG. 2A is a schematic transverse cross-sectional view of a foot orthosis that does not include a posting structure;

FIG. 2B is a schematic transverse cross-sectional view of a foot orthosis including a posting structure, in accordance with one exemplary embodiment;

FIG. 2C is a bottom view of a foot orthosis having a medial posting structure fabricated in accordance with one exemplary embodiment;

FIG. 2D is a bottom view of a foot orthosis having a lateral posting structure fabricated in accordance with one exemplary embodiment;

FIG. 3A provides a flow diagram of a method for designing and fabricating a customized foot orthosis, in accordance with an exemplary embodiment;

FIG. 3B provides a flow diagram of a method for generating a customized digital model of a foot orthosis, in accordance with an exemplary embodiment;

FIG. 4A is a representation of an initial digital model of a foot orthosis, in accordance with an exemplary embodiment;

FIG. 4B illustrates the heel region of the initial digital model of FIG. 4A;

FIG. 5A is an illustration of the peripheral portion of the surface profile of a posting structure formed on the heel region of the initial digital model of an orthosis, in accordance with an exemplary embodiment, the peripheral portion including a rear section and a front section;

FIG. 5B is an illustration of the determination of the salient portion of the surface profile of a posting structure based on the peripheral portion, where the salient portion is a line, in accordance with an exemplary embodiment;

FIG. 5C is an illustration of the determination of the salient portion of the surface profile of a posting structure based on the peripheral portion, where the salient portion is a surface region, in accordance with an exemplary embodiment;

FIG. 6 is an illustration of an example of a smoothing function that can be used to determine the first displacement parameter;

FIG. 7 depicts an initial digital model of a foot orthosis along with five customized digital models obtained by modifying the initial digital model. Each customized digital model includes a posting structure associated with a different pre-set posting height;

FIG. 8A is an illustration of an example of how local surface displacements can be computed based on the determination of the first and second displacement parameters;

FIG. 8B is a cross sectional transverse view of the orthosis of FIG. 8A taken along section line 8B in FIG. 8A, which illustrates a reference surface;

FIG. 9 is a bottom view of a heel region of a customized digital model of a foot orthosis, where the customized digital model has a medial posting structure without a heel notch, in accordance with one exemplary embodiment;

FIG. 10 is a bottom view of a heel region of a customized digital model of a foot orthosis, where the customized digital model has a bilateral posting structure without a heel notch, in accordance with one exemplary embodiment; and

FIG. 11 is schematic representation of an additive manufacturing system, in accordance with an exemplary embodiment.

### DETAILED DESCRIPTION

In the present description, similar features in the drawings have been given similar reference numerals. To avoid cluttering certain figures, some elements may not be indicated if they were already identified in a preceding figure. It should also be understood that the elements of the drawings are not necessarily depicted to scale, since emphasis is placed on clearly illustrating the elements and structures of the present embodiments. Furthermore, positional descriptors indicating the location and/or orientation of one element with respect to another element are used herein for ease and clarity of description. Unless otherwise indicated, these positional descriptors should be taken in the context of the figures and should not be considered limiting. It is appreciated that such spatially relative terms are intended to encompass different orientations in the use or operation of the present embodiments, in addition to the orientations exemplified in the figures. Furthermore, when a first element is referred to as being “on”, “above”, “below”, “over”, or “under” a second element, the first element can be either directly or indirectly on, above, below, over, or under the second element, respectively, such that one or multiple intervening elements may be disposed between the first element and the second element.

The terms “a”, “an”, and “one” are defined herein to mean “at least one”, that is, these terms do not exclude a plural number of elements, unless stated otherwise.

The term “or” is defined herein to mean “and/or”, unless stated otherwise.

Terms such as “substantially”, “generally”, and “about”, which modify a value, condition, or characteristic of a feature of an exemplary embodiment, should be understood to mean that the value, condition, or characteristic is defined within tolerances that are acceptable for the proper operation of this exemplary embodiment for its intended application or that fall within an acceptable range of experimental error. In particular, the term “about” generally refers to a range of numbers that one skilled in the art would consider equivalent to the stated value (e.g., having the same or an equivalent function or result). In some instances, the term “about” means a variation of +10% of the stated value. It is noted that all numeric values used herein are assumed to be modified by the term “about”, unless stated otherwise. The term “between” as used herein to refer to a range of numbers or values defined by endpoints is intended to include both endpoints, unless stated otherwise.

The term “based on” as used herein is intended to mean “based at least in part on”, whether directly or indirectly, and to encompass both “based solely on” and “based partly on”. In particular, the term “based on” may also be understood as meaning “depending on”, “representative of”, “indicative of”, “associated with”, and the like.

The terms “match”, “matching”, and “matched” refer herein to a condition in which two elements are either the same or within some predetermined tolerance of each other. That is, these terms are meant to encompass not only “exactly” or “identically” matching the two elements but also “substantially”, “approximately”, or “subjectively” matching the two elements, as well as providing a higher or best match among a plurality of matching possibilities.

The terms “connected” and “coupled”, and derivatives and variants thereof, refer herein to any connection or coupling, either direct or indirect, between two or more elements, unless stated otherwise. For example, the connection or coupling between elements may be mathematical, mechanical, optical, electrical, magnetic, thermal, chemical, logical, fluidic, operational, or any combination thereof.

The present description generally relates to foot orthotics and, more particularly, to computer-implemented techniques for designing a customized foot orthosis to include a posting structure, also known in the art as a stabilizer, which can be subsequently fabricated using 3D printing and other additive manufacturing processes. Non-limiting examples of possible fields of application include the generation of a model for a posting to be formed continuously and in one piece with an orthosis.

It is common practice to add various stabilizing orthotic elements, such as posting structures (also referred to as orthotic postings or simply postings), underneath a foot orthosis or orthotic insert to further block any undesired motion of the foot throughout the gait cycle, or alternatively guide the foot to a more desired position. These stabilizing elements are often comprised of a foam or rubber material that offers some compressibility, but they may also be made of a more rigid plastic or incorporated into the shell of the orthosis itself. Conventional orthotic postings can have various shapes and be provided at various positions, most commonly on the rearfoot or heel region of the orthosis, where they may be placed medially (inside), laterally (outside), or bilaterally (both sides) to control how the heel moves on heel strike. Furthermore, the “height” of the posting or the degree to which it blocks the motion can also be adjusted by changing the thickness of the applied material. Recent advancements in 3D scanning, 3D processing, and 3D printing technologies have enabled foot orthoses be reliability and repeatably 3D printed, while offering the same or comparable support and dynamic response as traditional orthoses.

As described in greater detail below, some embodiments disclosed herein rely on computer-aided techniques to design and fabricate foot orthoses with customized postings structures. In some embodiments, these customized foot orthoses can provide a more rounded, organic shape than those designed and fabricated by conventional approaches. Non-limiting examples of potential benefits or advantages provided by some embodiments disclosed herein include: a reduction of the abrupt impact on the patient's foot on the initial heel strike; an increase in the stiffness of the arms of the heel of the orthosis, resulting in an increase in the corrective force on the patient's heel generated intrinsically by the orthosis; a more gradual increase throughout the heel strike of the corrective force on the patient's heel (in the supination or pronation direction) generated by ground reaction forces from the orthosis, which in turn can allow the heel to be guided more gently into a desired position; and a better fit between the orthosis and the patient's shoe resulting from the more organic shape of the customized orthosis.

Various aspects, features and implementations of, or related to, the present techniques are described below with reference to the figures.

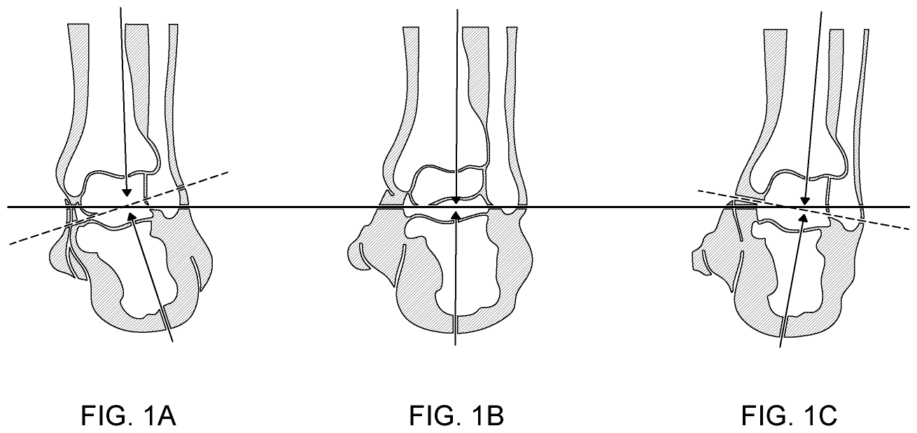

With reference to FIGS. 1A-1C, there is shown three positions of a foot. FIG. 1B shows the foot in a normal position, where the use of an orthotic may not be indicated. FIG. 1A and FIG. 1C display foot positions which may require correction through an orthotic device, such as an orthotic insert.

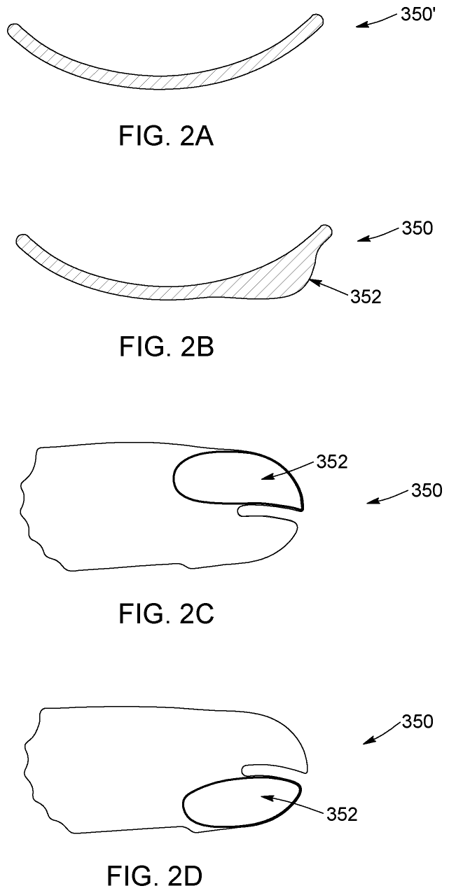

With reference to FIGS. 2A-2B, there is shown a schematic transverse cross-sectional view of a foot orthosis 350′ without (FIG. 2A) and a foot orthosis 350 with (FIG. 2B) a posting structure 352. The posting structure 352 of the foot orthosis 350 of FIG. 2B can designed and fabricated as part of the orthosis 350 and be shaped in accordance with an exemplary embodiment. FIGS. 2C-2D illustrate top down views of a foot orthosis 350 having a medial posting structure 352 (FIG. 2C) and an orthosis 350 having a lateral posting structure 352 in accordance with a respective embodiment.

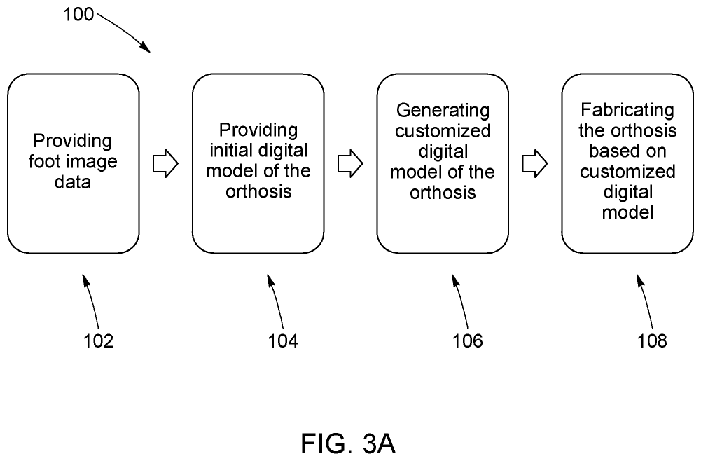

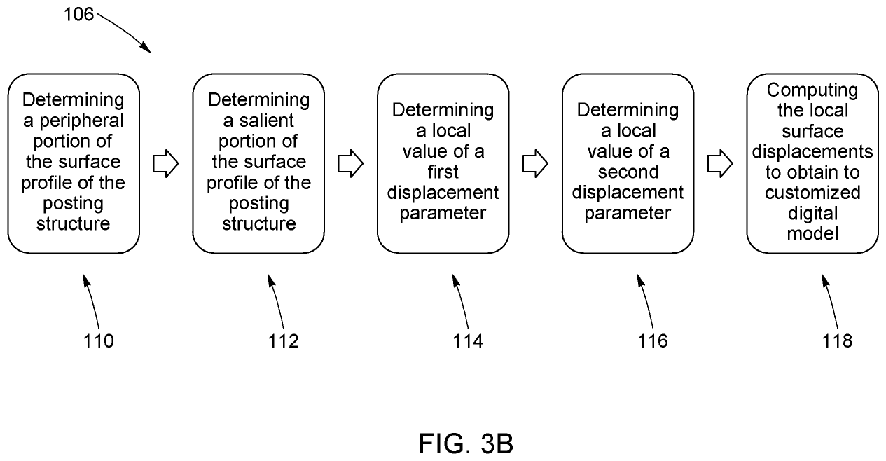

With reference to FIGS. 3A-3B, there is illustrated a flow diagram (FIG. 3A) of an embodiment of a method 100 of designing and fabricating a foot orthosis of a patient, and a flow diagram (FIG. 3B) of a possible implementation of step 106 of FIG. 3A of generating a customized digital model of the orthosis. The method 100 can be at least partially computer-implemented.

Referring to FIG. 3A, the method 100 can include a step 102 of providing foot image data of the foot, or part of the foot, of the patient, and a step 104 of providing an initial digital model of the orthosis based at least in part on the foot image data. The initial digital model is a digital model of the orthosis that does not include a posting structure. The method 100 can also include a step 106 of generating a customized digital model of the orthosis by improving or otherwise modifying the initial digital model to include a posting structure. The method 100 can further include a step 108 of fabricating the foot orthosis based on the customized digital model, for example, using additive manufacturing. More details regarding these and other possible steps of the method 100 are given below.

The term “foot image data” may include a digital image itself, or digital data that represents a digital image. In the present description, the expression “providing foot image data” is used broadly and can include, but is not limited to, making available for use, acquiring, obtaining, supplying, or receiving foot image data. By way of example, in some embodiments, the provision of the foot image data can involve the act of directly acquiring the foot image data (e.g., using an image capture device, such as a foot scanner) and making available for use the image data thus acquired. For example, acquiring the foot image data can include a step of scanning the foot of the patient to capture the foot image data as a topographical surface map or plantar image of the foot. In such a case, the foot image data may be provided, for example, using a scanning device such as the ones disclosed in co-assigned U.S. Pat. No. 9,757,035 and U.S. Patent Appl. Pub. No. 2019/0209093, the entire contents of which are incorporated herein by reference. Alternatively, any other imaging device which can provide a topographical surface map of a foot may be used. The topographical surface map of the foot may alternatively be said to include “3D image data” representative of the foot. The 3D image data would include, for example, an array of data points having coordinates corresponding to points on the foot, where each data point can be described by its spatial coordinate Pᵢ(xᵢ, yᵢ, zᵢ), where xᵢ, yᵢ and zᵢ are the cartesian coordinates of point i (Pᵢ), or an equivalent representation. The combination of these points would generate a surface representative of the surface of the foot. Depending on the application, the foot image data may be acquired in a non-weight-bearing condition, a full-weight-bearing condition, or a semi-weight-bearing condition.

In some embodiments, the step 104 of providing the initial digital model of the orthosis need not be made based on foot image data. In such a case, the step 102 of providing foot image data can be omitted, and any other means of obtaining an initial digital model of the orthosis may be used as deemed appropriate by the skilled addressee. For example, in some embodiments, the initial digital model can be selected from a virtual library or database containing a plurality of initial digital model templates. In some embodiments, the initial digital model can be determined based on a prescription of a clinician for correcting faulty movement, in combination or not with foot image data. It is appreciated that, in general, the initial digital model of the foot orthosis can be determined based on various techniques including, without limitation, empirical, analytical, numerical and experimental techniques, as well as on a combination of such techniques. Furthermore, depending on the application, the initial digital model can be a surface-based or a volume-based representation of the orthosis. The initial digital model can be provided in any suitable format.

In step 106, the initial digital model is converted to a customized digital model of the orthosis. The customized digital model includes, in addition to the orthosis itself, a posting structure. In some embodiments, the posting structure is formed on a heel region of the initial digital model. However, this is not a requirement, and forefoot, mid-foot, and rearfoot posting structures can be designed using the techniques described herein. In the case of rearfoot posting structures, the posting structure will generally be placed in a medial or a lateral region (or a combination of the two) of the heel region of the orthosis (i.e., in a rearward position of the orthosis). Whereas in conventional techniques postings would be manually and separately attached to the foot orthosis, the present techniques can allow for a customized digital model of an orthosis to be designed to include a posting structure and then be used to fabricate the orthosis itself, with the posting structure, through additive manufacturing.

It is an aim and advantage of some embodiments of the proposed method that the posting structure have a contoured shape, rather than a shape including flat edges. A contoured shape may, among other benefits, reduce abrupt impact of a patient's foot on a walking surface as the patient walks. With respect to FIGS. 2A and 2B, there are shown transverse cross-sectional views of an orthosis 350′ without (FIG. 2A) and an orthosis 350 with (FIG. 2B) a posting structure 352.

Referring more specifically to FIG. 3B, various aspects and features of the step 106 of generating the customized digital model by modifying the initial digital model to include the posting structure will now be described. The posting structure can generally be described in terms of its surface profile, which can be obtained by determining a set of local surface displacements to be applied to a corresponding set of surface points of the initial digital model. That is, the step 106 of generating the customized digital model involves a determination of the set of local surface displacements representing the posting structure. Broadly described, the determination of the set of local surface displacements can include steps 110, 112 of determining a peripheral portion (step 110) and a salient portion (step 112) of the surface profile of the posting structure, where the peripheral portion and the salient portion are respectively formed from a first subset and a second subset of the set of surface points of the initial digital model. The determination of the set of local surface displacements can also include steps 114, 116 of determining, for each surface point, a local value of a first displacement parameter P₁ (step 114) and a local value of a second displacement parameter P₂ (step 116), and a step 118 of computing each local surface displacement Dₛ from the local value of the first displacement parameter P₁ and the local value of the second displacement parameter P₁, so as to obtain the customized digital model.

The first displacement parameter P₁ is representative of the position of a given surface point with respect to the peripheral portion and the salient portion. The first displacement parameter P₁ is defined such that its value is minimum (typically zero) for the first subset of surface points (i.e., the surface points of the initial digital model corresponding to the peripheral portion of the posting structure) and maximum for the second subset of surface points (i.e., the surface points of the initial digital model corresponding to the salient portion of the posting structure). The first displacement parameter P₁ can be considered as an intrinsic displacement parameter, since its determination can be made based solely on the initial digital model, for example, by comparing, for each surface point, a degree of closeness to the peripheral portion to a degree of closeness to the salient portion. Furthermore, the first displacement parameter can be used to control the shape of the posting structure.

The second displacement parameter P₂ is representative of a distance of a given surface point from a reference surface associated with the initial digital model. For example, the reference surface can be a ground plane configured to contact a ground-contacting region of the initial digital model. In some embodiments, the method may involve a step of determining the reference surface, for example, by attempting to replicate, with the initial digital model, the placement of the orthosis on a flat surface (e.g., a table or a floor). In some embodiments, the reference surface may be defined as the lowest plane that touches the orthosis at the heel and at least one point (e.g., two points) at the forefoot. The reference surface may be tangent to the initial digital model at the point of contact with the heel, for example, if the reference surface is continuous (e.g., a non-uniform rational basis spline ( )urface), although this is not a requirement, for example, if the reference surface is discontinuous (e.g., a mesh). Alternatively, the reference surface may be a curved surface representing, for example, the insole of a shoe or another interfacing surface. The second displacement parameter P₂ can be considered as an extrinsic displacement parameter, since its determination is made based not only on the initial digital model, but also on elements that are external to the initial digital model, for example, by determining, for each surface point, a degree of closeness to the reference surface. Furthermore, the second displacement parameter P₂ can be used to control the size of the posting structure. More details regarding various aspects and features of steps 110, 112, 114, 116, and 118 are provided below.

To produce the customized digital model including the posting with a contoured shape, firstly a peripheral portion of the surface profile of the posting structure is determined (step 110 in FIG. 3B). The peripheral portion of the surface profile of the posting structure represents the boundary between the modified region (i.e., the region of the initial digital model enclosed by the peripheral portion, which contains the set of surface points to which the set of local surface displacements to be determined are to be applied) and the unmodified region of the initial digital model (i.e., the region of the initial digital model located outside the peripheral portion). It is appreciated that various approaches can be used to determine the peripheral portion of the surface profile of the posting structure including, without limitation, empirical, analytical, numerical and experimental techniques, as well as on a combination of such techniques. For example, in some embodiments, the peripheral portion can be selected from a virtual library or database containing a plurality of peripheral portion templates. In some embodiments, the peripheral portion can also or alternatively be determined based on a prescription of a clinician.

An exemplary manner of determining the peripheral portion of the surface profile of the posting structure will now be described in greater detail. Broadly described, the method generally includes a step of determining a rear section of the peripheral portion, and a step of determining a front section of the peripheral portion based on the rear section.

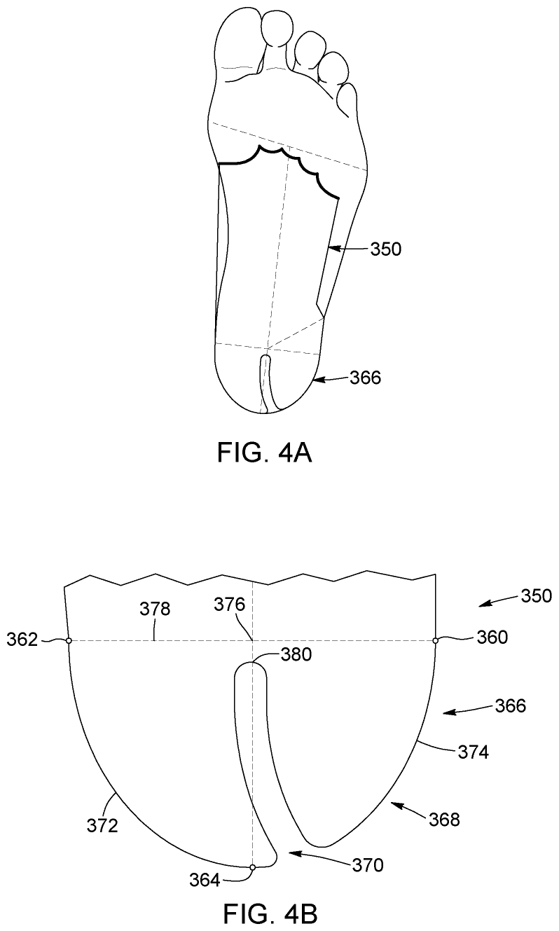

With reference to FIGS. 4A and 4B, there are shown views of an initial digital model of an orthosis 350, obtained in accordance with steps 102 and 104 of the method 100 as set out in FIG. 3A. The initial digital model of the orthosis 350 may be obtained based on a digital scan of a patient's foot and considering various prescribed components of the orthosis (e.g., heel width, forefoot width, etc.). The bottom surface of the initial digital model corresponds to the surface of the orthosis 350 that would be in contact, in use, with a ground plane. In the exemplary embodiment shown, the bottom surface is delimited by an outline of the orthosis 350. The heel region 366 of the initial digital model of the orthosis 350 may additionally be said to include three points, connecting which will generate a rear outline of the heel region 366 of the initial digital model. A lateral point 360 is positioned in a lateral portion of the heel region 366, a medial point 362 is positioned in a medial portion of the heel region 366, and a rearmost point 364 is positioned in a rearmost portion of the heel region 366.

In some embodiments, the step 110 of providing the initial digital model of the orthosis includes forming a heel notch 370 in the heel region 366 to split a rear contour 368 of the heel region 366 into a medial segment 372 and a lateral segment 374. In some embodiments, the provision of a heel notch 370 can allow control of the pronation/supination of the heel. In some embodiments, forming the heel notch 370 can include steps of determining, the rearmost point 364, the medial point 362, and a lateral point 360 in the heel region 366 of the initial digital model;

connecting the rearmost point 364 and the medial point 362 with a smooth curve, for example, a parabola, to generate the rear contour 368 of the heel region 366; and generating the heel notch 370 through the smooth curve to split the rear contour 368 into the lateral segment 374 and the medial segment 372. For example, as depicted in FIG. 4B, the rear contour 368 of the heel region 366 of the initial digital model of the orthosis 350 includes a heel notch 370 that begins laterally of the rearmost point 364 and that curves medially (the heel notch 370 may curve laterally in other embodiments) as it progresses forward toward the midpoint 376 of a line 378 connecting the lateral point 360 and the medial point 362. In some embodiments, by curving the heel notch 370 laterally or medially, one can alter the stiffness to push the heel towards a desired position. In one embodiment, the distance between the end 380 of the heel notch 370 and the midpoint 376 of the line 378 connecting the lateral point 360 and the medial point 362 may be of the order of a few millimeters, for example, about 4 mm. The heel notch 370 thus splits the heel portion into the medial segment 372 and the lateral segment 374.

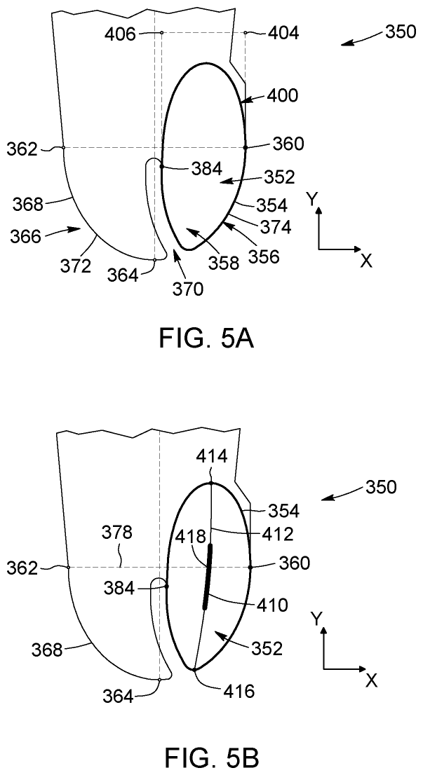

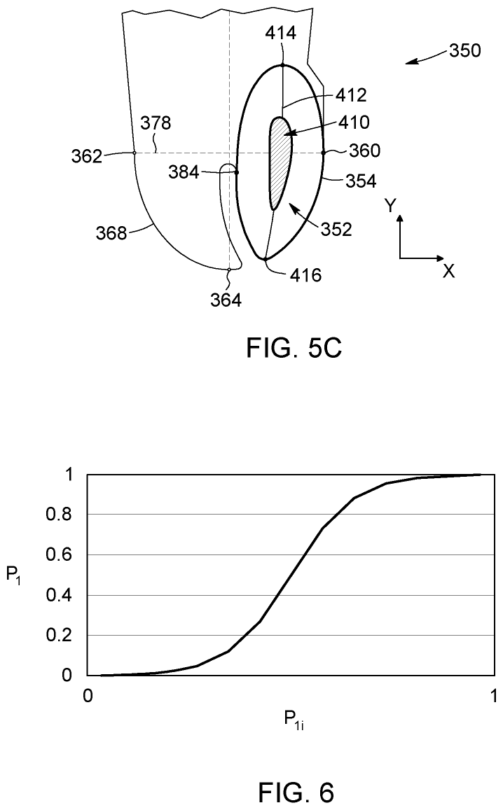

Turning to FIG. 5A, there is illustrated a peripheral portion 354 of the surface profile 358 of a posting structure 352 formed on the heel region 366 of the initial digital model of a orthosis 350, in accordance with an exemplary embodiment. The initial digital model illustrated in FIG. 5A corresponds to that depicted in FIGS. 4A and 4B. In FIG. 5A, the peripheral portion 354 includes a rear section 356 and a front section 400, and is depicted as a closed, thick solid line. In some embodiments, once the heel notch 370 has been provided, the determination of the rear section 356 of the peripheral portion 354 can include a step of forcing the rear section 356 to pass along part of the heel notch 370 and along part of either the lateral segment 374 (as in FIG. 5A; see the line connecting points 360 and 384 and defining the rear section 356 of the peripheral portion 354) or the medial segment 372 of the outline of the orthosis 350. Alternatively, the heel region 366 of the initial digital model may not have a notch.

Once the rear section 356 of the peripheral portion 354 of posting structure 352 has been determined, the front section 400 can be determined based on the rear section 356, resulting in a closed surface defining the peripheral portion 354 of the posting structure 352. In some embodiments, the front section 400 can be determined using a non-uniform rational basis spline (NURBS) curve defined by a set of control points, the set of control points including a first control point and a second control point corresponding to a first endpoint 360 and a second endpoint 384 of the rear section 356, respectively. With reference to FIG. 5A and in accordance with one exemplary embodiment, a third-degree NURBS curve is generated which connects the lateral point 360 to a proximal segment of the heel notch 370 at point 384 to form the front section 400 of the peripheral portion 354. The NURBS curve has a first endpoint and a second endpoint corresponding to points 360 and 384. The NURBS curve additionally has third and fourth control points corresponding to points 404 and 406 in FIG. 5A. In the illustrated example, point 406 is spaced apart from point 384 along the Y Axis but has the same X coordinate (i.e., X₄₀₆=X₃₈₄). Point 404 is spaced apart from point 360 along the Y Axis but has the same X coordinate (i.e., X₄₀₄=X₃₆₀). In the illustrated example, the following exemplary relationship is also satisfied: Y ₄₀₆=Y ₄₀₄=1.2×(Y₃₆₀−Y₃₆₄)+Y₃₈₄. Although third-degree NURBS curves are commonly used, other types of NURBS curves may also be used. Alternatively, any other type of curves or lines may be used, such as an arc, a straight line or a combination thereof, to close the curve of the peripheral portion 354 at the front section 400 thereof (i.e., to determine the front section 400 of the peripheral portion 354 from the rear section 356). In one embodiment, the curve 402 defining the front section 400 of the peripheral portion 354 may be a parabola.

Alternatively, the peripheral portion 354 may be determined by other means. For example, the peripheral portion 354 may be custom drawn by a technician during design of the orthosis 350 using Computer Aided Design (CAD) software. Although in the displayed example the peripheral portion 354 has a largely oval shape, other shapes may be desired or prescribed. For example, the posting structure 352 may instead have a largely rectangular shape. Furthermore, while the illustrated embodiment includes a peripheral portion 354 which is at least partially coincident with an outer contour of the initial digital model of the orthosis 350, the peripheral portion 354 may also be entirely inwardly offset from the outer contour of the initial digital model of the orthosis 350, corresponding to the rear contour 368 of heel region 366, so as not to be coincident with the rear outline of the orthosis 350.

Returning briefly to FIG. 3B, the step 112 of determining the salient portion of the surface profile of the posting structure will now be described in greater detail. In some embodiments, the salient portion may be determined based on the peripheral portion, and may include the following steps: determining a frontmost point and a rearmost point of the peripheral portion of the surface profile of the posting structure; creating a line extending between the frontmost point and the rearmost point of the peripheral portion; and defining the salient portion to correspond at least in part to an intermediate portion of the line.

An exemplary implementation of these steps is depicted in FIG. 5B. where the salient portion 410 (thick solid line) extends at least in part along an intermediate portion of the line 412 which runs from a frontmost point 414 of the peripheral portion 354 to a rearmost point 416 of the peripheral portion 354 and which includes the X-midpoint 418 of the line connecting points 360 and 384. The line passing through points 414, 416, and 418 can be obtained, for instance, using a third-degree interpolated NURBS curve. In this example, the salient portion 410 corresponds to the middle third of the line connecting points 414 and 416 through point 418. The salient portion 410 defines the portion of the posting structure 352 having the greatest surface displacement relative to the initial digital model with respect to the first displacement parameter P₁. The exact amount of displacement (i.e., in absolute value) is determined by the second displacement parameter P₂, which can be based on the prescription and/or the patient's need for corrective support on the orthosis. Although in the illustrated embodiment the salient portion 410 extends along a line, it could alternatively extend across a closed curve to create a larger surface for the posting structure 352 to contact a ground surface. Reference is made to, for example, to FIG. 5C, where the salient portion 410 is defined as the region inside the hatched closed curve. Additionally, although in the illustrated embodiment of FIG. 5B the salient portion 410 only has a slight curve connecting the frontmost point 414 with the rearmost point 416, the salient portion 410 may alternatively connect two points at any other positions along the peripheral portion 354 or have a curve with a 90-degree angle so that the salient portion 410 is largely L-shaped. Other variations are equally envisaged.

The surface profile of the posting structure 352 connects the peripheral portion 354 to the salient portion 410 and can be obtained by determining a set of local surface displacements to the set of surface points of the initial digital model that are located on or within the peripheral portion 354. The determination of the set of local surface displacements can be carried out by considering two criteria, namely a first displacement parameter P₁ and a second displacement parameter P₂.

Returning briefly to FIG. 3B, the step 114 of determining a local value of the first displacement parameter P₁ for each surface point of the set of surface points of the initial digital model will be described in greater detail. In some embodiments, this step 114 can include, for each surface point, a step of determining an initial value of the first displacement parameter, Pᵢ. For example, the initial value of the first displacement parameter P₁ᵢ of each surface point can be determined using a distance ratio expressed by the formula:

$${P}_{1 i} = \frac{{D}_{Peripheral}}{{D}_{Peripheral} + {D}_{Salient}}$$

Where:
Dₚₑᵣᵢₚₕₑᵣₐₗ=Shortest distance of the surface point under consideration from the peripheral portion, and
D_{Salient}=Shortest distance of the surface point under consideration from the salient portion.

The initial value of the first displacement parameter, P₁ᵢ, has a minimum value of zero on the peripheral portion 354 (corresponding to the first subset of the set of surface points of the initial digital model; see above) and reaches a maximum value of one on the salient portion 410 (corresponding to the second subset of the set of surface points of the initial digital model; see above). Each of the remaining surface points of the initial digital model thus have a local value of P₁ᵢ that is greater than 0 but less than 1. The initial value of the first displacement parameter P₁ᵢ therefore defines the location of a point along the posting surface with respect to the peripheral and salient portions. That is, the closer a given surface point is to the peripheral (salient) portion, the closer to 0 (1) its local value of P₁ᵢ will be.

In some embodiments, the initial value of the first displacement parameter P₁ᵢ can be then processed through a smoothing function to yield the first displacement parameter P₁. Without a smoothing function, the first displacement parameter P₁ would linearly increase from the minimum value of 0 on the peripheral portion to the maximum value of 1 on the salient portion in accordance with the distance ratio function given above. By using a smoothing function, the first displacement parameter P₁ may instead follow any other desired curve to provide control over the shape of the posting structure. For example, the initial first displacement parameter P₁ᵢ may be passed through a sigmoid function (e.g., the error function erf(x) and the logistic function) to generate a smoother curve, such as the curve in FIG. 6. Alternatively, the smoothing function need not be a sigmoid function but may have any other suitable shape. In other embodiments, the smoothing may be accommodated by a superposition of the smoothing function and the general behavior of a NURBS surface if NURBS control points are being modified. This is because modifying the control points of a NURBS surface or curve will cause the underlying surface or curve to be smoothly or continuously modified.

It is appreciated that the use of a smoothing function can ensure or help ensure smoother transitions for the surface points located close to the peripheral portion (which defines the boundary between the affected and unaffected areas of the initial digital model) or close to the salient portion. It is also appreciated that, in some embodiments, a smoothing function need not be used in the determination of the first displacement parameter P₁, in which case P₁ᵢ=P₁. It is further appreciated that the distance ratio function Dₚₑᵣᵢₚₕₑᵣₐₗ/(Dₚₑᵣᵢₚₕₑᵣₐₗ+Dₛₐₗᵢₑₙₜ) is provided by way of example only, and that various functions involving Dₚₑᵣᵢₚₕₑᵣₐₗ and Dₛₐₗᵢₑₙₜ can be used to compare the degree of closeness to the peripheral portion to the degree of closeness to the salient portion.

Returning briefly to FIG. 3B, the step 116 of determining a local value of the second displacement parameter P₂ for each surface point of the set of surface points of the initial digital model will be described in greater detail. In some embodiments, the reference surface with respect to which the second displacement parameter P₂ is determined can be a ground plane configured to contact a ground-contacting region of the initial digital model, although other reference surfaces can be used in other embodiments. In some embodiments, the determination of the second displacement parameter P₂ can involve, for each surface point, a step of determining a distance of the surface point from the ground plane, and a step of setting the local value of the second displacement parameter P₂ equal to a fraction of the distance of the surface point to the ground plane. In such embodiments, the local value of the second displacement parameter P₂ can depend on two variables, the distance to the ground from the surface point under consideration, and a fraction or percentage value corresponding to a desired posting height of the posting structure:

$${P}_{2} = {D}_{r} \cdot R$$

Where:
Dᵣ=Distance from the surface point under consideration to a reference surface, and
R=Posting height percentage.

It is appreciated that while the first displacement parameter P₁ may be determined with reference to the initial digital model alone (i.e., the peripheral and salient portions), the second displacement parameter P₂ is determined with respect to a reference surface. The reference surface may, for example, be the insole of a shoe into which the orthosis is inserted. The reference surface may also be a ground plane in contact with a ground contacting region of the initial digital model. The second displacement parameter P₂ further includes a posting height percentage R. The posting height percentage R is a percentage value corresponding to the desired posting height.

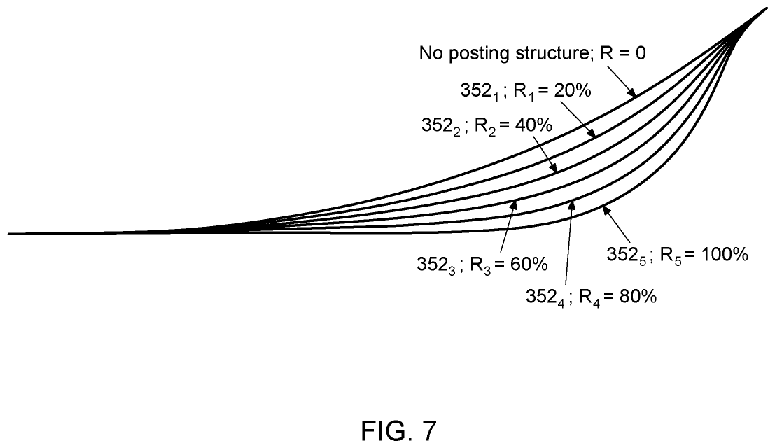

In some embodiments, the selection of the posting height percentage R can be made from a plurality of pre-set posting height percentages, the pre-set height percentages being the distance of a surface point to the ground plane. The pre-set height percentages could be stored in a virtual library stored on a non-transitory storage medium and range from 0-100% in, for example, 20% increments. As illustrated in FIG. 7, an initial digital model corresponding to a 0% posting height percentage (i.e., no posting structure) is illustrated, along with five customized digital models modified from the initial digital model to include posting structures 352₁-352₅ characterized by five different posting-height percentage values R₁-R₅ equal to 20%, 40%, 60%, 80%, and 100%, respectively. In some embodiments, the selected pre-set posting height percentage is the same for all the surface points to be displaced, while in other embodiments, it can vary across the set of surface points.

Returning to FIG. 3B, the step 118 of computing the local surface displacement of each surface point to obtain the customized digital model will be described in greater detail. The local surface displacement Dₛ of each surface point can be determined based on the local values of the first and second displacement parameters P₁, P₂ according to the following formula:

$${D}_{s} = {P}_{2} \cdot {P}_{1}$$

Depending on the application, the local surface displacement Dₛ applied to each surface point of the initial digital model to reach its final position and form the posting structure can be applied in the direction of the local surface normal or along any suitable direction (e.g., a direction perpendicular to a ground-contacting plane associated with the orthosis).

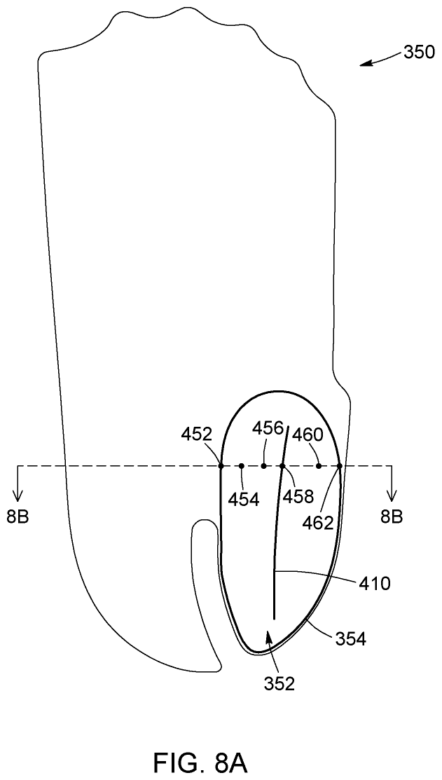

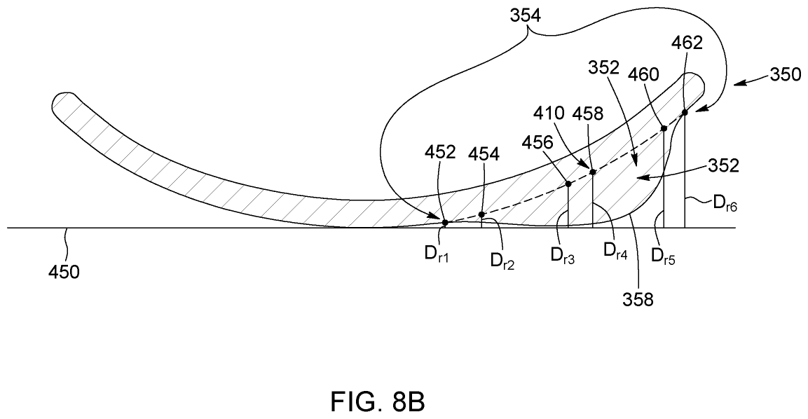

Referring now to FIGS. 8A and 8B, an example of how local surface displacements Dₛ can be computed based on the determination of the first and second displacement parameters P₁, P₂ will be described. FIG. 8A is a bottom view illustrating how a customized digital model of an orthosis 350 including a posting structure 352 can be obtained by modifying an initial digital model of the orthosis 350. The posting structure 352 includes a peripheral portion 354 and a salient portion 410, which can be determined such as described above. In this embodiment, the peripheral portion 354 of the posting structure 352 is entirely inwardly offset from the outer contour of the orthosis 350. In some embodiments, offsetting the peripheral portion 354 of the posting structure 352 from the outer contour of the orthosis 350 can facilitate merging the posting structure 352 with the orthosis 350. Alternatively, the part of the peripheral portion 354 may be coincident with the outer contour of the orthosis 350, as in FIGS. 5A-5C. FIG. 8B is a transverse cross-sectional view of FIG. 8A taken along section line 8B, which further illustrates a reference plane 450 (e.g., a ground plane) associated with the orthosis 350. In the illustrated example, six surface points 452, 454, 456, 458, 460, 462 of the initial digital model are considered, whose respective local surface displacements Dₛ are to be determined to generate the posting structure 352 and obtain the customized digital model. Surface points 452 and 462 are located on the peripheral portion 354 of the surface profile 358 of the posting structure 352 (i.e., first subset of surface points), so that their local values of the first displacement parameter, P₁, are equal to 0 (minimum value of P₁). Surface point 458 is located on the salient portion 410 (e.g., second subset of surface points), so that its local value of P₁, is equal to 1 (maximum value of P₁). Surface points 454 and 460 are closer to the peripheral portion 354 than to the salient portion 410 by the same degree, so that their local values of P₁ are the same, and set equal to 0.1, for definiteness. Finally, surface point 456 is closer to the salient portion 410 than to the peripheral portion 354, and its local value of P₁ is set equal to 0.9, for definiteness. These values of P₁ are summarized in Table 1 below.

Turning to FIG. 8B, the distance Dᵣ₁-Dᵣ₆ of each surface point 452-462 from the reference surface 450 increases from surface point 452 to surface point 462. For definiteness, six exemplary values for Dᵣ are given in Table 1. Table 1 also lists, for each of the six surface points 452-462, the local value of the second displacement parameter P₂, obtained using the relationship above (i.e., P₂=Dᵣ×R, with the posting height percentage R set to 0.75, for definiteness), and the local surface displacement Dₛ=P₁×P₂ to be applied to the respective surface point 452-462 to form the posting structure 352 and generate the customized digital model. It is appreciated that, as expected, surface points 452 and 462 each have a value of local surface displacement Dₛ equal to zero, since they are located on the peripheral portion 354 of the posting structure352.

TABLE 1
Calculations of local surface displacements Ds from the
first and second displacements parameters P₁ and P₂.

| Surface | | | | | | Local surface | |
|---|---|---|---|---|---|---|---|
| point of | | | | Second | | displacement | |
| initial | First | Distance to | | displacement | | Dₛ to be | |
| digital | displacement | reference | | parameter P₂ | | applied to | |
| model | parameter P₁ | surface Dᵣ | | (for R = 0.75) | | surface point | |
| 452 | 0.0 | 5 | mm | 3.75 | mm | 0.0 | mm |
| 454 | 0.1 | 8 | mm | 6 | mm | 0.6 | mm |
| 456 | 0.9 | 10 | mm | 7.5 | mm | 6.75 | mm |
| 458 | 1 | 15 | mm | 11.25 | mm | 11.25 | mm |
| 460 | 0.1 | 25 | mm | 18.75 | mm | 1.875 | mm |
| 462 | 0.0 | 30 | mm | 22.5 | mm | 0.0 | mm |

The above values were determined using a unique posting height percentage value of 0.75, though other values could equally be used. In practice, the customized digital model can be generated by computing the local surface displacements Dₛ for a significantly larger number of surface points on or within the peripheral portion 354. For example, in some embodiments, the number of surface points can range from about 50 to about 5000, depending on the posting size and mesh resolution or control point density, as appropriate. The greater the number of points where the first and second displacement parameters (and deformation values) are calculated, the greater the resolution of the resulting posting surface profile. There may be a minimum and maximum number of points where the first and second displacement parameters P₁, P₂ (and deformation value) are calculated to provide the desired resolution of posting surface profile without excess computational burden.

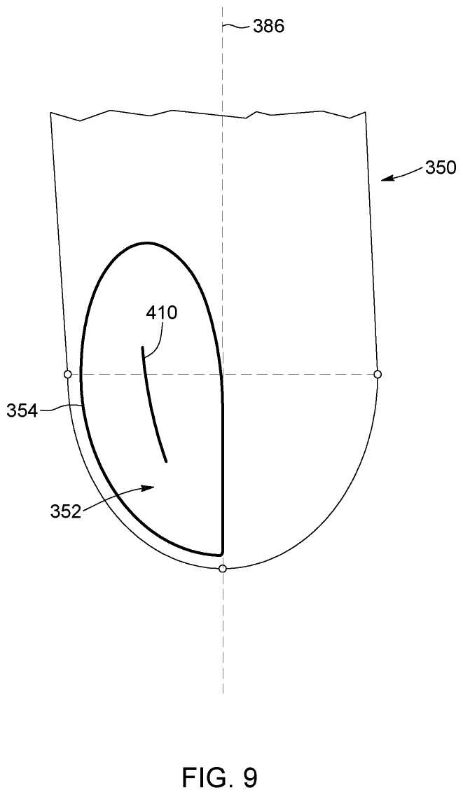

Referring to FIG. 9, there is shown another embodiment of a customized digital model of an orthosis 350, which can be obtained in accordance with the method 100 of FIG. 3A. The customized digital model illustrated in FIG. 9 includes a medial posting structure 352 and does not include a heel notch. The posting structure 352 includes a peripheral portion 354 and a salient portion 410, which can be defined using techniques similar to those described above. The peripheral portion 354 of the posting structure 352 is laterally offset from the outer contour of the orthosis 350. Furthermore, the peripheral portion 354 is partially coincident with an axial midline 386 of the orthosis 350. As described above, the surface profile of the posting structure 352 of the customized digital model of FIG. 9 can be obtained by applying a set of local surface displacements to a corresponding set of surface points of an initial digital model of the orthosis 350, where each local surface displacement is determined from a first and a second displacement parameter

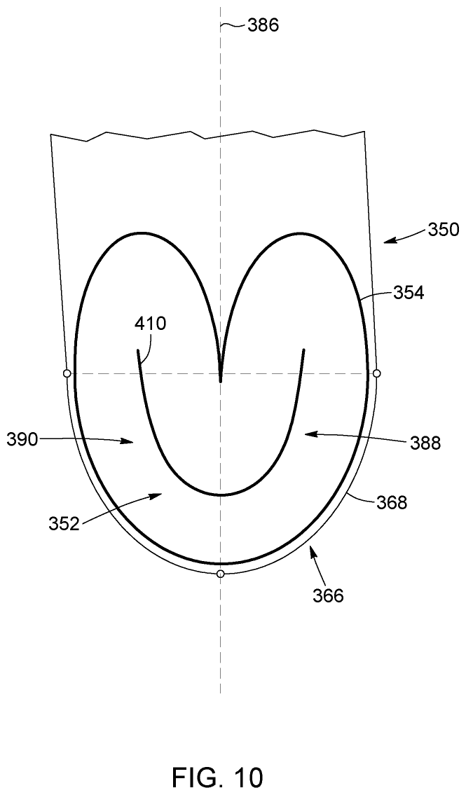

Referring to FIG. 10, there is shown another embodiment of a customized digital model of an orthosis 350, which can be obtained in accordance with the method 100 of FIG. 3A. The customized digital model illustrated in FIG. 10 includes a bilateral posting structure 352. The bilateral posting structure 352 includes a peripheral portion 354 and a salient portion 410, which can be defined using techniques as described above. In FIG. 10, the peripheral portion 354 and the salient portion 410 both extend laterally and medially of a midline 386 of the orthosis 350, such that the posting structure 352 includes both a lateral portion 388 and a medial portion 390. As described above, the surface profile of the posting structure 352 of the customized digital model of FIG. 10 can be obtained by applying a set of local surface displacements to a corresponding set of surface points of an initial digital model of the orthosis 350, where each local surface displacement is determined from a first and a second displacement parameter.

The total height of the posting structure of the orthosis can, in one embodiment, be adjusted to account for leg length discrepancy where one leg has a different length than the other. Accordingly, the orthosis of one foot can have a thickness that is different from that of the orthosis of the other foot to account for this discrepancy, as well as to correct posture. A bilateral posting structure may provide improved balance in such cases where leg length discrepancy is corrected, relative to an orthosis having a strictly medial or lateral posting structure. Although the embodiment of FIG. 10 does not have a heel notch, it is understood that such a notch may be provided to obtain an orthosis having a bilateral posting structure in accordance with the previously described methods.

Returning to FIG. 3A, once the customized digital model has been generated (step 106), the method 100 can include an additive manufacturing operation of fabricating the foot orthosis based on the customized digital model (step 108). In such embodiments, the foot orthosis may be fabricated as a single integral item, with the posting structure being fully and smoothly incorporated with the rest of the orthosis. In one embodiment, the posting structure is made of the same material as the rest of the orthosis. In an alternative embodiment, the posting is made of a different material from the rest of the orthosis. For example, the posting structure could be made of a softer material in one embodiment, or alternatively of a more rigid material depending on the prescription and needs of the patient. Although in one embodiment the orthosis is fabricated using additive manufacturing techniques (e.g., 3D printing), other techniques may be envisaged. For example, the proposed method may be used to create a mold to create the orthosis with the posting structure using an injection molding technique. The orthotic could equally be fabricated using subtractive manufacturing techniques, for example, by milling from a plastic block or another suitable material.

According to another aspect, there is provided a non-transitory computer readable storage medium or memory storing a computer program or executable instructions thereon that, when executed by a computer or processor, can perform various steps of the method of designing an orthosis, such as disclosed herein.

In the present description, the terms “computer readable storage medium” and “computer readable memory” are intended to refer to a non-transitory and tangible computer product that can store and communicate executable instructions for the implementation of various steps of the methods disclosed herein. The computer readable memory may be any computer data storage device or assembly of such devices, including a random-access memory (RAM); a dynamic RAM; a read-only memory (ROM); a magnetic storage device, such as a hard disk drive, a solid state drive, a floppy disk, and a magnetic tape; an optical storage device, such as a compact disc (CD or CDROM), a digital video disc (DVD), and a Blu-Ray™ disc; a flash drive memory; and/or any other non-transitory memory technologies. A plurality of such storage devices may be provided, as can be appreciated by those skilled in the art. The computer readable memory may be associated with, coupled to, or included in a computer or processor configured to execute instructions contained in a computer program stored in the computer readable memory and relating to various functions associated with the computer.

According to another aspect, there is provided a computer device including a processor and a non-transitory computer readable storage medium such as disclosed herein, the non-transitory computer readable storage medium being operatively coupled to the processor.

In the present description, the term “processor” should not be construed as being limited to a single processor, and accordingly, any known processor architecture may be used. In some implementations, the processor may include a plurality of processing units. Such processing units may be physically located within the same device, or the processor may represent processing functionality of a plurality of devices operating in coordination. For example, the processor may include or be part of a computer; a microprocessor; a microcontroller; a coprocessor; a central processing unit (CPU); an image signal processor (ISP); a digital signal processor (DSP) running on a system on a chip (SoC); a single-board computer (SBC); a dedicated graphics processing unit (GPU); a special-purpose programmable logic device embodied in hardware device, such as, for example, a field-programmable gate array (FPGA) or an application-specific integrated circuit (ASIC); and/or any other device configured to electronically process information and to operate collectively as a processor.

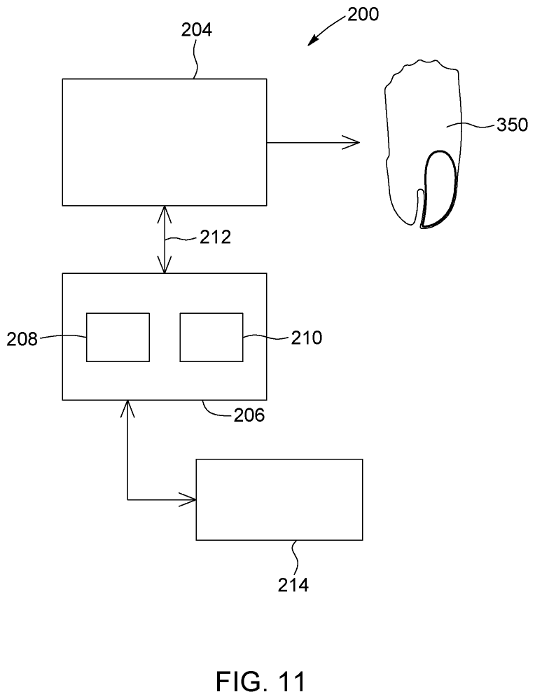

Referring to FIG. 11, according to another aspect, there is provided an additive manufacturing system 200 for designing a foot orthosis 350. The additive manufacturing system 200 generally includes an additive manufacturing device 204 and a computer device 206. The computer device 206 can include a processor 208 and a non-transitory computer readable storage medium 210 having stored thereon computer readable instructions that, when executed by the processor 208, cause the processor 208 to perform a method of generating a customized digital model 212 of the foot orthosis 350 from an initial digital model, where the customized digital model 212 includes a posting structure. The computer device 206 is operatively coupled to the additive manufacturing device 204 and configured to control the additive manufacturing device 204 to manufacture the foot orthosis 350 based on the customized digital model 212. In some embodiments, the additive manufacturing system 200 may also include an imaging device 214, such as a 3D scanner, operatively coupled to the computer device 206. The imaging device 214 may be configured to acquire foot image data of the foot of the patient, and the computer device 206 may be configured to receive the foot image data and provide therefrom an initial digital model of the orthosis 350.

Numerous modifications could be made to the embodiments described above without departing from the appended claims.

1. A method of designing an orthosis for a foot of a patient, the method comprising:
providing an initial digital model of the orthosis; and
generating a customized digital model of the orthosis by modifying the initial digital model to include a posting structure, the posting structure having a surface profile obtained by applying a set of local surface displacements to a corresponding set of surface points of the initial digital model, said generating comprising:
determining a peripheral portion of the surface profile of the posting structure, the peripheral portion being formed from a first subset of the set of surface points of the initial digital model;
determining a salient portion of the surface profile of the posting structure, the salient portion being formed from a second subset of the set of surface points of the initial digital model;
determining, for each surface point, a local value of a first displacement parameter, the first displacement parameter being representative of a position of the surface point with respect to the peripheral portion and the salient portion of the surface profile of the posting structure, wherein the first subset of surface points is associated with a minimum value of the first displacement parameter and the second subset of surface points is associated with a maximum value of the first displacement parameter;
determining, for each surface point, a local value of a second displacement parameter, the second displacement parameter being representative of a distance of the surface point from a reference surface associated with the initial digital model; and
computing, for each surface point, the local surface displacement based on the local value of the first displacement parameter and the local value of the second displacement parameter, thereby generating the customized digital model.

2. The method of claim 1, wherein determining, for each surface point, the local value of the first displacement parameter comprises:
determining an initial value of the first displacement parameter; and
applying a smoothing function to the initial value to obtain the local value of the first displacement parameter.

3. The method of claim 2, wherein the smoothing function is a sigmoid curve.

4. The method of claim 2, wherein determining, for each surface point, the initial value of the first displacement parameter comprises:
determining a distance ratio of the shortest distance of the surface point from the peripheral portion to a sum of the shortest distance of the surface point from the peripheral portion and the shortest distance of the surface point from the salient portion; and
using the distance ratio as the initial value of the first displacement parameter.

5. The method of claim 1, wherein the reference surface associated with the initial digital model is a ground plane in contact with a ground-contacting region of the initial digital model.

6. The method of claim 5, wherein determining, for each surface point, the local value of the second displacement parameter comprises:
determining a distance of the surface point from the ground plane; and
setting the local value of the second displacement parameter equal to a fraction of the distance of the surface point from the ground plane.

7. The method of claim 6, further comprising providing a virtual library containing a plurality of pre-set posting height percentages and selecting, from the virtual library, a selected one of the plurality of pre-set posting height percentages as the fraction of the distance of the surface point from the ground plane.

8. The method of claim 7, wherein the selected pre-set posting height percentage is the same for all the surface points of the set of surface points of the initial digital model.

9. The method of claim 1, wherein the peripheral portion of the surface profile of the posting structure coincides, along a portion thereof, with an outer contour of the initial digital model of the orthosis.

10. The method of claim 1, wherein the peripheral portion of the surface profile of the posting structure is entirely inwardly offset from an outer contour of the initial digital model of the orthosis.

11. The method of claim 1, wherein the posting structure is formed on a heel region of the initial digital model.

12. The method of claim 11, wherein determining the peripheral portion of the surface profile of the posting structure comprises:
determining a rear section of the peripheral portion; and
determining a front section of the peripheral portion based on the rear section.

13. The method of claim 12, wherein:
providing the initial digital model of the orthosis comprises forming a heel notch in the heel region to split a rear contour of the heel region into a medial segment and a lateral segment; and
determining the rear section of the peripheral portion comprises forcing the rear section to pass along part of the heel notch and along part of either the lateral or the medial segment, or both the lateral segment and the medial segment.

14. The method of claim 13, wherein forming the heel notch comprises:
determining, a rearmost point, a medial point, and a lateral point in the heel region of the initial digital model;
connecting the rearmost point to the medial point with a curve to generate the rear contour of the heel region; and
generating the heel notch through the curve to split the rear contour of the heel region into the lateral segment and the medial segment.

15. The method of claim 14, wherein the curve is a parabola.

16. The method of claim 12, wherein the front section of the peripheral portion is determined using a non-uniform rational basis spline (NURBS) curve defined by a set of control points, the set of control points comprising a first control point and a second control point corresponding to a first endpoint and a second endpoint of the rear section, respectively.

17. The method of claim 1, wherein the peripheral portion of the posting structure is partially coincident with a midline formed through the initial digital model of the orthosis.

18. The method of claim 1, wherein the peripheral portion of the posting structure extends laterally and medially of a midline formed through the initial digital model of the orthosis.

19. The method of claim 18, wherein the salient portion of the posting structure extends laterally and medially of the midline.

20. The method of claim 18, wherein the peripheral portion of the posting structure comprises a straight line, an arc segment, a NURBS, or a combination thereof, passing through the midline.

21. The method of claim 1, wherein the salient portion of the surface profile of the posting structure is determined based on the peripheral portion.

22. The method of claim 21, determining the salient portion of the surface profile of the posting structure comprises:
determining a frontmost point and a rearmost point of the peripheral portion of the surface profile of the posting structure;
creating a line extending between the frontmost point and the rearmost point of the peripheral portion; and
defining the salient portion to correspond at least in part to an intermediate portion of the line.

23. The method of claim 1, further comprising an additive manufacturing operation of fabricating the orthosis based on the customized digital model.

24. The method of claim 1, further comprising providing foot image data of the foot of the patient, and wherein providing the initial digital model of the orthosis comprises determining the initial digital model based on the foot image data.

25. The method of claim 24, wherein providing the foot image data comprises scanning the foot to capture the foot image data as a topographical surface map of the foot.

26. A non-transitory computer readable storage medium having stored thereon computer readable instructions that, when executed by a processor, cause the processor to perform a method of designing an orthosis for a foot of a patient according to claim 1.

27. A computer device comprising:
a processor and
the non-transitory computer readable storage medium of claim 26, the non-transitory computer readable storage medium being operatively coupled to the processor.

28. An additive manufacturing system for designing an orthosis for a foot of a patient, the additive manufacturing system comprising:
an additive manufacturing device; and
the computer device of claim 27, wherein the computer device is operatively coupled to the additive manufacturing device and configured to control the additive manufacturing device to manufacture the orthosis based on the customized digital model.

29. The additive manufacturing system of claim 28, further comprising an imaging device operatively coupled to the computer device, the imaging device being configured to acquire foot image data of the foot of the patient, wherein the computer device is configured to receive the foot image data and provide therefrom the initial digital model of the orthosis.
